(19) United States
(12) Patent Application Publication
(10) Pub. No.: US 2026/0106003 A1
(43) Pub. Date: Apr. 16, 2026

(54) METHOD AND SYSTEM FOR MEDICAL RECORD PROCESSING AND DISPLAY

(71) Applicant: Ajith Kumar Perakathu Somasekharan, Griffith (AU)

(72) Inventor: Ajith Kumar Perakathu Somasekharan, Griffith (AU)

(21) Appl. No.: 19/115,556

(22) PCT Filed: Sep. 26, 2023
(86) PCT No.: PCT/IB2023/059486

§ 371 Date: Mar. 26, 2025

(30) Foreign Application Priority Data
Sep. 26, 2022 (AU) 2022902783

(51) Int. Cl. G16H 10/60; G06T 13/40

(52) U.S. Cl. CPC G16H 10/60; G06T 13/40

(57) ABSTRACT

Disclosed is a system configured to display medical information about a patient, the system comprising: a processor configured to process a medical record for the patient and assign information from the medical record to one of a plurality of medical categories; and a display for displaying a 3D representation of the patient, the 3D representation including a 3D avatar of the patient having corresponding regions for each of the plurality of medical categories, wherein, the processor receives an indication that a user has selected one of the regions and sends to the display information assigned to the corresponding medical category.

### TECHNICAL FIELD

The present disclosure relates to processing and displaying medical record data for a patient.

### BACKGROUND

Medical records for a patient have developed from a traditional paper based filing system. Under a paper based filing system, each patient would have a file used to collect and store relevant entries. For a medical surgery, entries could be added each time new information for the patient was recorded. Each entry, such as information relating to a visit by a patient, could be entered into the file with a separate piece of paper. Due to the nature of the system, linking between entries was an operation for the clinician entering the data in the file.

Such paper-based system have now been replaced by electronic medical record systems which can manage large volumes of data for a patient. Patient data may be arranged in a raw data manner that mimics a paper-based system, lacking any links between patient data entry. Such an unlinked representation of the data may provide little assistance to a GP or specialists when they attempt to retrieve or get accurate insights into the complex information of the patient's history provided by the electronic record. Gaining accurate insights may also be limited due to appointment lengths between a doctor and a patient. While more time may provide greater insights into the medical history of a patient, such time may not be available or may be expensive.

The amount of time required to accurately review a complete medical record may increase dramatically for a patient with a complex medical history. This may be the case where the electronic medical record is chronologically ordered text histories with records spanning multiple entries made by different clinicians over many years.

In addition to reviewing a medical history, a clinician must also interact with a patient during an appointment. As a result, the clinician may not be able to commit all of their attention and focus on the review of the medical file, making the medical file review more difficult as interpreting a lengthy patient history and locating relevant information for the current consultation may be time consuming.

### SUMMARY

This Summary is provided to introduce a selection of concepts in a simplified form that are further described below in the Detailed Description. This Summary is not intended to identify essential features of the claimed subject matter, nor is it intended to be used to limit the scope of the claimed subject matter.

Disclosed is a system configured to display medical information about a patient, the system comprising: a processor configured to process a medical record for the patient and assign information from the medical record to one of a plurality of medical categories; and a display for displaying a 3D representation of the patient, the 3D representation including a 3D avatar of the patient having corresponding regions for each of the plurality of medical categories, wherein, the processor receives an indication that a user has selected one of the regions and sends to the display information assigned to the corresponding medical category.

Also disclosed is a system wherein at least one of the medical categories is a biological system.

Also disclosed is a system wherein the medical categories are selected from the set consisting of: skeletal, muscular, cardiovascular, nervous system, endocrine, lymphatic, respiratory, digestive, urinary, mental health and reproductive.

Also disclosed is a system wherein the regions for the medical categories are representative regions of the 3D avatar.

Also disclosed is a system wherein the 3D representation is a layered architecture model of the 3D avatar.

Also disclosed is a system wherein the 3D representation changes to a layered architecture model of the 3D avatar.

Also disclosed is a system wherein the layered architecture model includes at least one layer selected from the set of: skin, muscular, organs and skeletal.

Also disclosed is a system wherein at least one of the regions is associated with at least one layer of the layered architecture model.

Also disclosed is a system wherein the 3D avatar changes to the layered architecture model based on input from the user.

Also disclosed is a system wherein the at least one of the regions associated with the at least one layer is further divided in subregions.

Also disclosed is a system wherein each of the subregions is associated with part of the at least one layer.

Also disclosed is a system wherein the at least one layer shows an icon to represent a diagnosis.

Also disclosed is a system wherein the diagnosis is an ICD (International Statistical Classification of Diseases and Related Health Problems).

Also disclosed is a system wherein the information assigned to the corresponding medical category is an ICD.

Also disclosed is a system wherein the information assigned to the corresponding medical category is a treatment plan.

Also disclosed is a system wherein the information assigned to the corresponding medical category is a treatment outcome.

Disclosed is a computer implemented method of displaying medical information, the method comprising: receiving a medical record for a patient; assigning information from the medical record to one of a plurality of medical categories; displaying a 3D avatar for the patient, the 3D avatar including corresponding regions for each of the plurality of medical categories displayed on the 3D avatar; and displaying the assigned information for one of the plurality of medical categories when a user selects the corresponding region.

Also disclosed is a computer implemented method wherein each of the medical categories is a biological system.

Also disclosed is a computer implemented method wherein the medical categories are selected from the set consisting of: skeletal, muscular, cardiovascular, nervous system, endocrine, lymphatic, respiratory, digestive, urinary, mental health and reproductive.

Also disclosed is a computer implemented method wherein the regions for the medical categories are representative regions of the 3D avatar.

Also disclosed is a computer implemented method wherein the 3D representation is a layered architecture model of the 3D avatar.

Also disclosed is a computer implemented method wherein the displaying further comprises: replacing the 3D representation with a layered architecture model of the 3D avatar.

Also disclosed is a computer implemented method wherein the layered architecture model includes at least one layer selected from the set of: skin, muscular, organs and skeletal.

Also disclosed is a computer implemented method wherein at least one of the regions is associated with at least one layer of the layered architecture model.

Also disclosed is a computer implemented method wherein the 3D avatar is replaced by the layered architecture model based on input from the user.

Also disclosed is a computer implemented method wherein the at least one of the regions associated with the at least one layer is further divided in subregions.

Also disclosed is a computer implemented method wherein each of the subregions is associated with part of the layer.

Also disclosed is a computer implemented method wherein the at least one layer shows an icon to represent a diagnosis.

Also disclosed is a computer implemented method wherein the diagnosis is an ICD (International Statistical Classification of Diseases and Related Health Problems).

Also disclosed is a computer implemented method wherein the information assigned to the corresponding medical category is an ICD.

Also disclosed is a computer implemented method wherein the information assigned to the corresponding medical category is a treatment plan.

Also disclosed is a computer implemented method wherein the information assigned to the corresponding medical category is a treatment outcome.

### BRIEF DESCRIPTION OF FIGURES

At least one embodiment of the present invention is described, by way of example only, with reference to the accompanying figures.

FIG. 1 illustrates a functional block diagram of an example processing system that can be utilised to embody or give effect to a particular embodiment;

FIG. 2 illustrates an example network infrastructure that can be utilised to embody or give effect to a particular embodiment;

FIGS. 3A and 3B illustrate a 3D patient avatar of a medical record processing system according to a particular embodiment;

FIG. 4 illustrates an overview of a medical record processing system according to a particular embodiment;

FIG. 5 illustrates an overview of an avatar generator according to a particular embodiment;

FIG. 6 illustrates components of an example AI engine of the medical record processing system;

FIG. 7 illustrates a communications overview of an example medical record processing system according to a particular embodiment;

FIG. 8 illustrates a 3D avatar process of the medical record processing system according to a particular embodiment;

FIG. 9 illustrates a medical record processing system according to a particular embodiment; and

FIG. 10 illustrates a medical record processing method of a medical record processing system according to a particular embodiment.

### DETAILED DESCRIPTION

The following modes, given by way of example only, are described in order to provide a more precise understanding of one or more embodiments. In the figures, like reference numerals are used to identify like parts throughout the figures.

The disclosed medical record processing system processes medical records to generate a 3D avatar showing a visual representation of medical records of a patient. The medical record processing system uses a medical assistant, also referred to as an intelligent assistant, to process electronic medical records using artificial intelligence. Information from the processed medical records are assigned, or classified, according to a medical category. The medical category may be a biological system, body part, a region of the body, an ICD (International Statistical Classification of Diseases and Related Health Problems), a treatment plan or a treatment outcome. Medical categories may be associated with a region of a 3D avatar displayed to a user. When the user selects the region, information assigned to the corresponding medical category may be displayed. The regions for the medical categories may be overlaid on top of biological system of the 3D avatar for the user to select. The medical record processing system provide clinicians visual data for the patient and may help provide clinicians with information to make decisions using a patient centric data model.

Disclosed is a system configured to display and manage clinical information, such as medical information, about a patient. The system may comprise a processor configured to process a medical record for the patient and assign information from the medical record to one of a plurality of medical categories. The system may also include a display for displaying a 3D representation of the patient, the 3D representation including a 3D avatar of the patient having corresponding regions for each of the plurality of medical categories. The processor can receive an indication that a user has selected one of the regions and display information assigned to the corresponding medical category

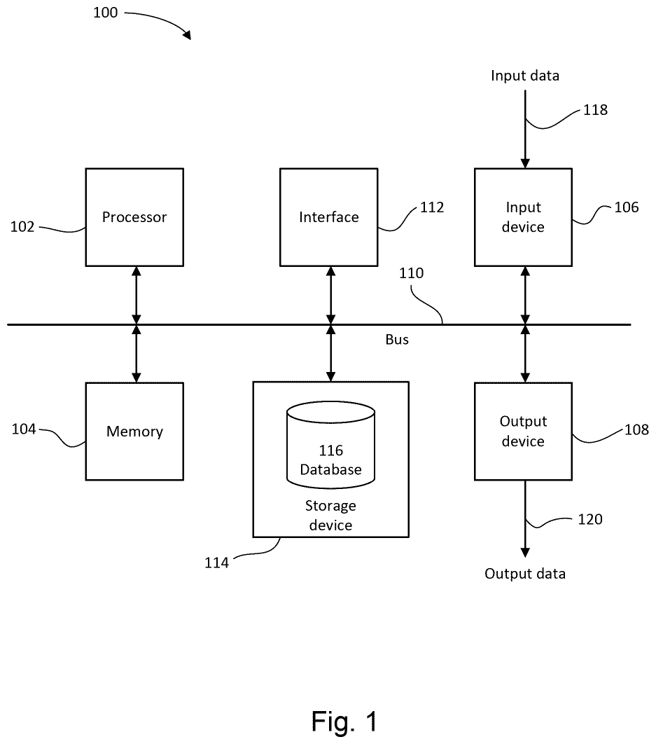

A particular embodiment of the present invention can be realised using a processing system, an example of which is shown in FIG. 1. In particular, the processing system 100 generally includes at least one processor 102, or processing unit or plurality of processors, memory 104, at least one input device 106 and at least one output device 108, coupled together via a bus or group of buses 110. In certain embodiments, input device 106 and output device 108 could be the same device. An interface 112 can also be provided for coupling the processing system 100 to one or more peripheral devices, for example interface 112 could be a PCI card or PC card. At least one storage device 114 which houses at least one database 116 can also be provided. The memory 104 can be any form of memory device, for example, volatile or non-volatile memory, solid state storage devices, magnetic devices, etc. The processor 102 could include more than one distinct processing device, for example to handle different functions within the processing system 100.

Input device 106 receives input data 118 and can include, for example, a keyboard, a pointer device such as a pen-like device or a mouse, audio receiving device for voice controlled activation such as a microphone, data receiver or antenna such as a modem or wireless data adaptor, data acquisition card, etc. Input data 118 could come from different sources, for example keyboard instructions in conjunction with data received via a network. Output device 108 produces or generates output data 120 and can include, for example, a display device or monitor in which case output data 120 is visual, a printer in which case output data 120 is printed, a port for example a USB port, a peripheral component adaptor, a data transmitter or antenna such as a modem or wireless network adaptor, etc. Output data 120 could be distinct and derived from different output devices, for example a visual display on a monitor in conjunction with data transmitted to a network. A user could view data output, or an interpretation of the data output, on, for example, a monitor or using a printer. The storage device 114 can be any form of data or information storage means, for example, volatile or non-volatile memory, solid state storage devices, magnetic devices, etc.

In use, the processing system 100 is adapted to allow data or information to be stored in and/or retrieved from, via wired or wireless communication means, the at least one database 116. The interface 112 may allow wired and/or wireless communication between the processing unit 102 and peripheral components that may serve a specialised purpose. The processor 102 receives instructions as input data 118 via input device 106 and can display processed results or other output to a user by utilising output device 108. More than one input device 106 and/or output device 108 can be provided. It should be appreciated that the processing system 100 may be any form of terminal, server, specialised hardware, or the like.

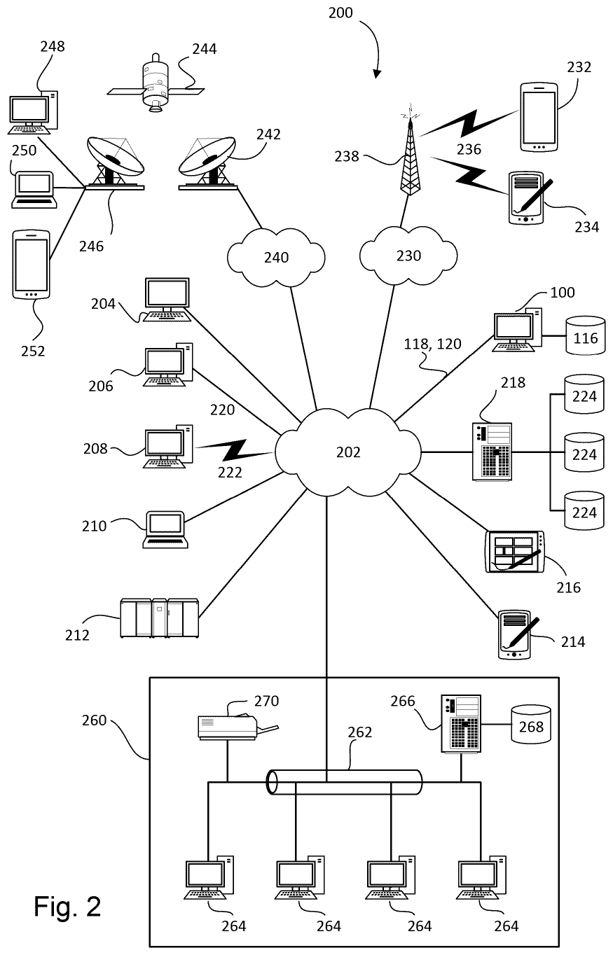

The processing system 100 may be a part of a networked communications system 200, as shown in FIG. 2. Processing system 100 could connect to network 202, for example the Internet or a WAN. Input data 118 and output data 120 could be communicated to other devices via network 202. Other terminals, for example, thin client 204, further processing systems 206 and 208, notebook computer 210, mainframe computer 212, PDA 214, pen-based computer or tablet 216, server 218, etc., can be connected to network 202. A large variety of other types of terminals or configurations could be utilised. The transfer of information and/or data over network 202 can be achieved using wired communications means 220 or wireless communications means 222. Server 218 can facilitate the transfer of data between network 202 and one or more databases 224. Server 218 and one or more databases 224 provide an example of an information source.

Other networks may communicate with network 202. For example, telecommunications network 230 could facilitate the transfer of data between network 202 and mobile, cellular telephone or smartphone 232 or a PDA-type device 234, by utilising wireless communication means 236 and receiving/transmitting station 238. Satellite communications network 240 could communicate with satellite signal receiver 242 which receives data signals from satellite 244 which in turn is in remote communication with satellite signal transmitter 246. Terminals, for example further processing system 248, notebook computer 250 or satellite telephone 252, can thereby communicate with network 202. A local network 260, which for example may be a private network, LAN, etc., may also be connected to network 202. For example, network 202 could be connected with ethernet 262 which connects terminals 264, server 266 which controls the transfer of data to and/or from database 268, and printer 270. Various other types of networks could be utilised.

The processing system 100 is adapted to communicate with other terminals, for example further processing systems 206, 208, by sending and receiving data, 118, 120, to and from the network 202, thereby facilitating possible communication with other components of the networked communications system 200.

Thus, for example, the networks 202, 230, 240 may form part of, or be connected to, the Internet, in which case, the terminals 206, 212, 218, for example, may be web servers, Internet terminals or the like. The networks 202, 230, 240, 260 may be or form part of other communication networks, such as LAN, WAN, ethernet, token ring, FDDI ring, star, etc., networks, or mobile telephone networks, such as GSM, CDMA, 4G, 5G etc., networks, and may be wholly or partially wired, including for example optical fibre, or wireless networks, depending on a particular implementation.

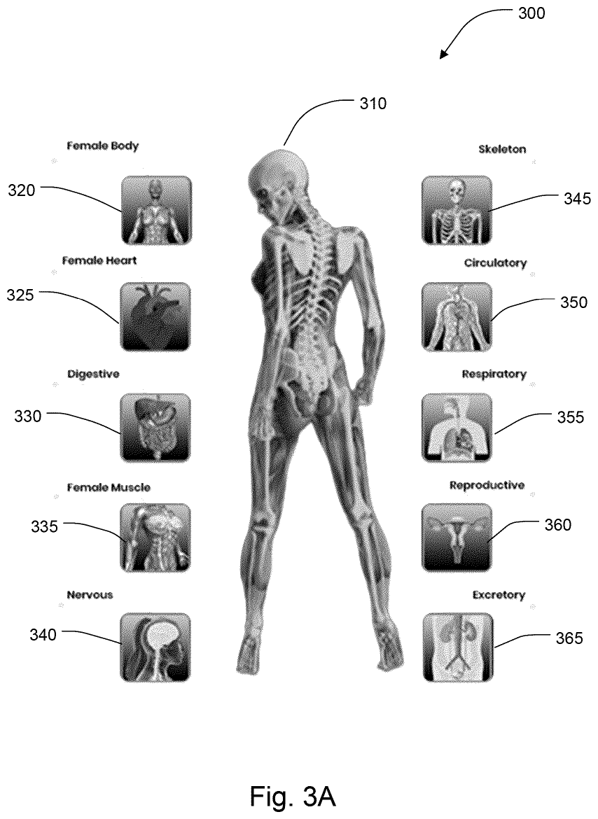

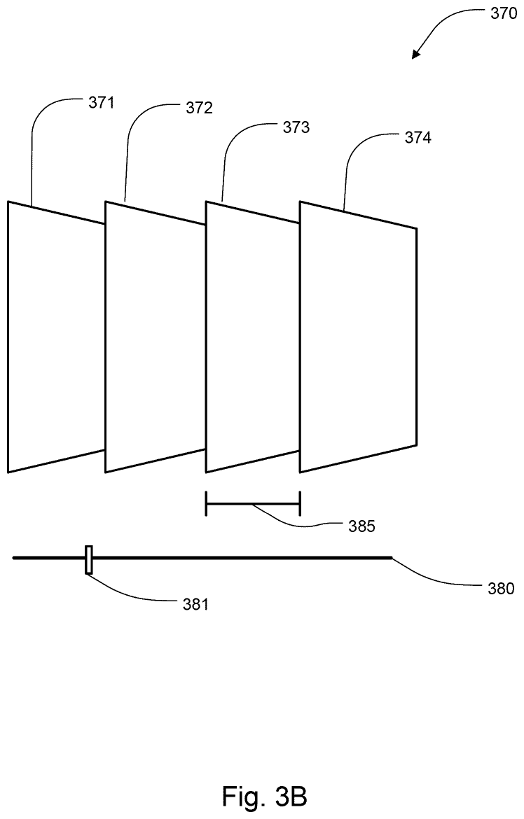

FIG. 3A shows a 3D patient history 300 that may be displayed to a user of the medical record processing system. The 3D patient history 300 may be generated by the processing system 100. The information displayed on the 3D patient history 300 may be prepared and stored in advance on the processing system 100. The medical record may include information, also known as patient data, such as family history, social history, previous diagnosis notes, other reports and a history of the patient presentations.

The 3D patient history 300 shows a 3D avatar 310 that is a graphical representation of a body of the patient. Information relating to the patient is presented to the user of the system and grouped by medical categories. The medical categories shown for the 3D avatar 310 are body 320, heart 325, digestive 330, muscles 335, nervous 340, skeleton 345, circulatory 350, respiratory 355, reproductive 360 and excretory 365. Each of the medical categories has a selection region that may provide information related to the medical category when the region is selected by the user. In one example, the displayed selection regions may change colour or show an additional icon to indicate that the medical category associated with the region contains further information about the patient. The additional icon may also be used to show diagnosis information where the medical record processing system may select use predetermined icons to convey the diagnosis information. The icon may display an icon to represent an ICD (International Statistical Classification of Diseases and Related Health Problems) as the diagnosis. By selecting the region, such as the digestive 330, further information may be presented from the medical record relating to the patient's digestive system. Information from the electronic medical record is processed and collated by the medical record processing system to allow rapid access by the user based on medical categories. Each of the selectable regions may indicate patient data is available to provide a patient history in a pictorial manner, which may allow clinicians to understand the patient history quickly. Each selectable region may indicate further information can be presented, from the medical record, for the medical category record associated with selection region.

While the example 3D patient history 300 shows selectable regions for the medical categories, separate to the 3D avatar 310, the selectable regions may be integrated into the 3D avatar 310. In such an example, the heart 325 may be displayed as a selectable region over a heart of the 3D avatar 310. In this example, regions of the 3D avatar 310 may use an indicator, such as colours, size, shape and/or intensity change, to indicate selectable regions of the 3D avatar 310 or may change an indicator on an event such as a mouse over. The indicators of the 3D avatar 310 may also change to show that more information is present or to attract attention of the user, depending on the usability model of the medical record processing system. In one example, the 3D patient history 300 may change to a layered display, such as the layered display described in relation to FIG. 3B, when more information is present.

FIG. 3B shows a layered 3D avatar 370 where a 3D avatar, such as the 3D avatar 310 of FIG. 3A may be split into separate layers to show a biological structure of a human. The layered 3D avatar 370 has a skin layer 371, a muscular layer 372, an organ layer 373 and a bone layer 374. Each of the layers may show a detailed data model of the 3D avatar, graphically representing each layer. For example, the bone layer 374 may show a skeletal representation, the organ layer 373 may show only the organs of the 3D avatar, the muscular layer 372 may show a muscular representation without skin, and the skin layer 371 just show skin. Typically, the skin layer 371 will look like a person.

The layers of the layered 3D avatar 370 may be controlled by a layer separation slider 380 which adjusts a distance between each of the layers. In other examples, the layer separation may be controlled using voice command or selecting the layers to cycles between a plurality of pre-set distances. A user of the medical record processing system may move a layer separation marker 381 to change a layer gap 385 between the layers. In one example, moving the layer separation marker 381 to the far left of the layer separation slider 380 will recombine all the layers into a single 3D avatar. Manipulation of the layer separation marker 381 may change the 3D avatar from a single representation, such as the 3D avatar 310, to a layered representation such as the layered 3D avatar 370. As a result, the 3D avatar 310 changes to the layered 3D avatar 370 based on input from the user.

Selection regions may be placed on different layers. For example, the bone layer 374 may provide information on bone health of the patient. The medical record processing system may also change where selection regions are positioned based on the present of the layered 3D avatar 370. For the bone layer 374, information on specific bones may be available based on a selection subregion associated with the bone. If a patient has broken a bone in their left leg, then a selection subregion on the bone may be selected by the user to bring up x-ray information for the bone or other information such as when the break occurred. Similarly, information about muscles may be mapped to selection subregions of the muscular layer 372, information about organs may be mapped to selection subregions of the organ layer 373 and skin information mapped to selection subregions of the skin layer 371. Use of the layered 3D avatar 370 may allow a user of the system to subdivide selection regions from of the 3D avatar 310 into subregions of the layered 3D avatar 370.

The layers of the layered 3D avatar 370 may also show patient data including diagnosed ICDs or other diagnosis information. The patient data may be displayed as an icon, from a predetermined set of icons, or the patient data may be displayed in response to the user selecting a region. In one example the patient data displayed on a layer may be layer relevant information selected based on the other information on the layer. For example, patient data related to muscles may be displayed on the muscular layer 372, information relating to skin displayed on the skin layer 371, information relating to organs on the organ layer 373 and information relating to bones on the bone layer 374. Information relating to each ICD diagnosis report, treatment effectiveness, and reports may be displayed, or available, to the user.

FIG. 3B may allow a clinician to look at internal items on the 3D avatar, such as organ details, body parts, etc. on the layers. The user interface may provide attachable and detachable options for the internal items. With detachable and attachable options, the layered 3D avatar 370 may allow a user to view related data such as pathology and radiology by displaying extra information. The user may select what information is viewable by applying a filter to select internal items of the layer, or 3D avatar that are viewable. In one example, the user may use a voice command to display only information related to cardiology. In another example, the user may select information from a list where items selected by the user determines what information is displayed. The detachable and attachable options may apply to internal items and/or medical categories and may be applied to the 3D avatar, such as the 3D avatar 310, or a layered presentation such as the layered 3D avatar 370.

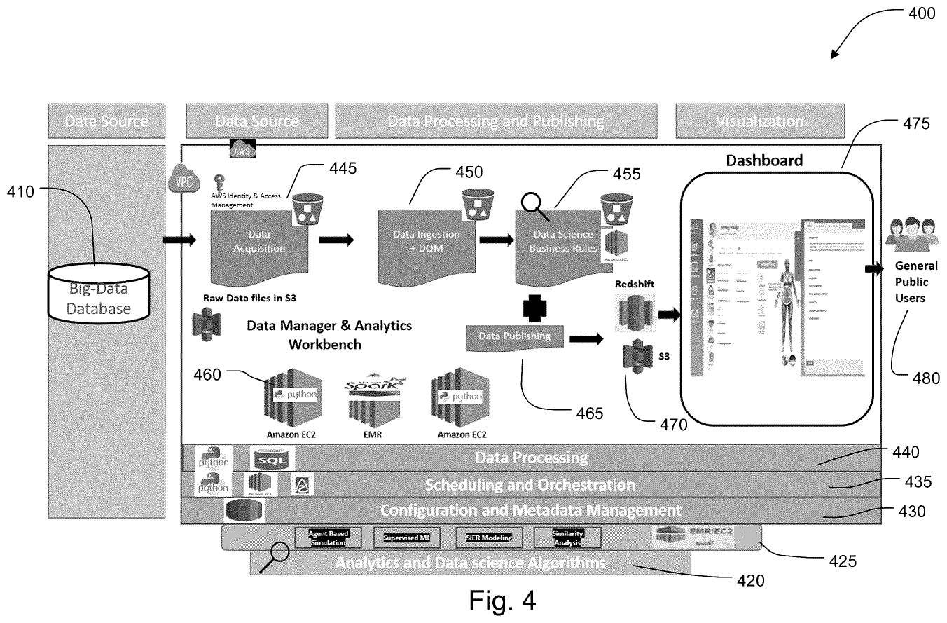

A medical record processing system overview 400 will be described in relation to FIG. 4. The medical record processing system overview 400 may be practiced on a computer such as the processing system 100 communicating over a network 202 or, as shown in FIG. 4, on a number of computers communicating over a network. The medical record processing system overview 400 takes patient information from medical records for the patient, processes the medical records and displays information from the medical records to a user, allowing the user to explore the medical record using a 3D avatar.

The medical record processing system overview 400 includes a database 410 that may store medical records. The medical records may be processed by processing modules 420 which includes artificial intelligence processes 425, configuration processes 430, scheduling processes 435 and data processes 440. The processing modules 420 may be used at different stages of the medical record processing system overview 400 to process data.

Data from the database 410 is read by a data acquisition process 445 that queries the database 410 to select relevant data. In one example, the data read by the data acquisition process 445 is data from a medical record of a patient. The information may be selected at the start of a consultation between a clinician and the patient. Next a data processing process 450 takes the data from the data acquisition process 445 and may apply predetermined rules to correct the data and apply data quality measures to ensure that the data from the database 410 is in a standardised from for later processing.

At a data transformation process 455, the data from the data processing process 450 is modified or transformed using some of the processing modules 420. Some of the processing of the data transformation process 455 is described below and may include assigning, or classifying, information from the medical record of the patient to one of a plurality of medical classification. At a data output 465 the processed data is published. At a data storage 470, the processed data from the data transformation process 455 may be stored for later use, such as subsequent consultation between the user and the patient. The data output 465 may be sent to a display 475 where a 3D avatar of the patient is used to display information relating to the patient. The displayed information may include selectable regions that a user 480 may select using a user interface. Each of the regions may have a corresponding medical category associated with the region and selection of the region will present to the user information from the medical record of the patient that was assigned to the medical category at the data transformation process 455.

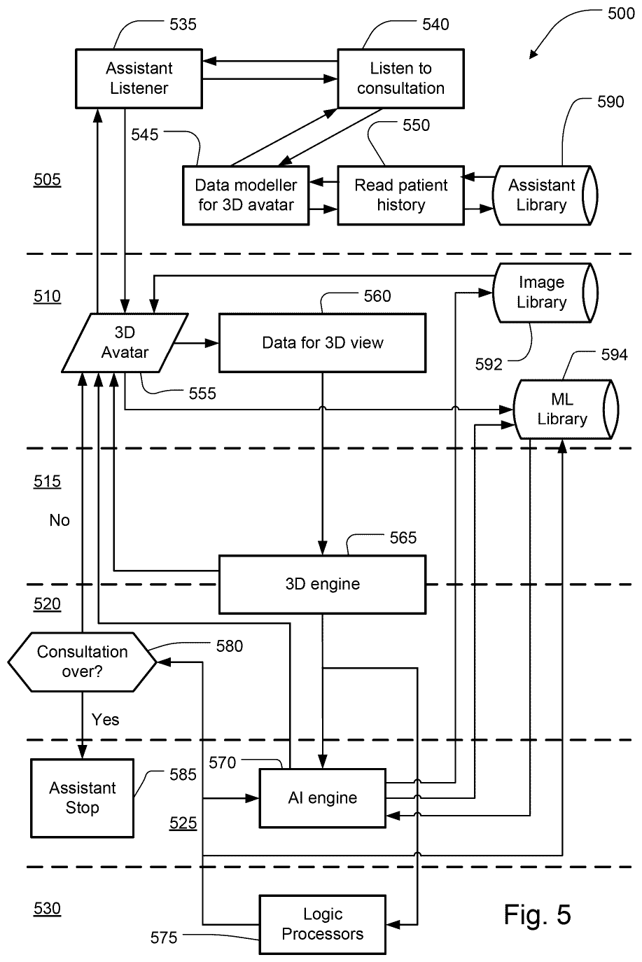

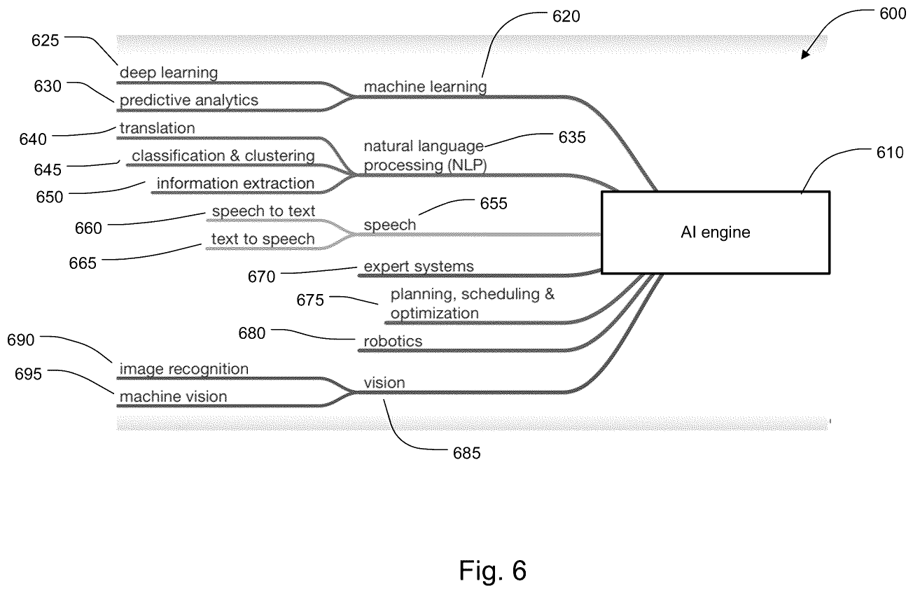

An avatar generator overview 500 will now be described in relation to FIG. 5. The avatar generator overview 500 shows components of the medical record processing system that may generate a display of the 3D patient avatar. The avatar generator overview 500 is broken down into the following components, a listener 505, intelligent agent 510, ICD Engine 515, dictionaries 520, AI engine 525 and logic processors 530. Each of the levels may provide processes or data not shown in FIG. 5. Further information on the AI engine 525 is provided in relation to FIG. 6.

The avatar generator overview 500 may be practiced on a computer such as the processing system 100 communicating over a network 202. The avatar generator overview 500 is an example of how a medical record processing system may be implemented. The avatar generator overview 500 may show a subset of connections and process used for operation of the medical record processing system. In one example, data may be passed between modules, such as processes, engines, and libraries of the avatar generator overview 500 using a data bus. In this example, all of the modules are connected to the data bus and can send or request data from other modules via the data bus.

At the listener 505, the avatar generator overview 500 has an assistant listener 535 that may operate as a process collecting information about a patient for display to a user. The listener 535 communicates to a consultation input process 540 that can listen to a consultation between a clinician and a patient, if the medical record processing system is granted access to a microphone. The listener 505 also has a data modeller for a 3D avatar process 545 that can receive patient history from a read history process 550. The read history process 550 can read information from an assistant library 590 that may store information such as patient categories. The read history process 550 takes input from a medical record database and processes text or image components, as well as information from the consultation input process 540, to provide data to the data modeller for 3D avatar process 545 for display on 3D avatar. The data modeller for 3D avatar process 545 takes output from the read history process 550 and processes the output in preparation for display to the user. In one example, the data modeller for the 3D avatar process 545 may use AI processing from an AI engine 570 to assign the output from the read history process 550 to one of a predetermined number of medical categories. In one example, the data modeller for 3D avatar process 545 may connect directly to a 3D avatar process 555 to transfer information that forms part of the 3D avatar. In another example, the output of the data modeller for 3D avatar process 545 may be sent to a 3D view data 560

As shown, the listener 505 has four processes and a library. In one example of the avatar generator overview 500 the four processes are combined into a single process that performs operations of the assistant listener process 535, the consultation input process 540, the data modeller for 3D avatar process 545 and the read history process 550.

The intelligent agent 510 includes the 3D avatar process 555, the 3D view data 560, an image library 592 and a machine learning library 594. The 3D avatar process 555 takes input of patient data from the data modeller for 3D avatar process 545, images from the image library 592 to form part of the 3D avatar, data from a 3D engine 565 and output from the AI engine 570. The 3D avatar process 555 outputs the 3D view data 560 used by the 3D engine process 565 to construct the 3D avatar for display to a user of the medical record processing system. The 3D avatar process 555 may update the machine learning library 594 with information that may be used to update or train parts of the machine learning modules in the AI engine 570.

The AI engine 570, and other processes, may be executed on logic processors 575. The logic processors 575 perform data management at a framework level and may transfer learning models to other engines or components of the avatar generator overview 500. The AI engine 570 executes one or more machine learning modules that can be used to execute, or assist components such as the assistant listener process 535, the data modeller for 3D avatar process 545 and the 3D avatar process 555. Component may be loaded from the machine learning library 594. In one example of the avatar generator overview 500, the AI engine 570 may execute a machine learning module, with a configuration for the machine learning module loaded from the machine learning library 594.

Once a consultation between a clinician and a patient is finished, the avatar generator overview 500 may progress to a consultation over 580 where a check is made with a user to end the consultation. If the consultation is not over then the avatar generator overview 500 continues to operate, such as returning to the 3D engine 565. If the consultation is finished, then the avatar generator overview 500 may proceed to an end 585.

The ICD Engine 515 may process information received from the medical record of the patient to generate an ICD (International Statistical Classification of Diseases and Related Health Problems) code. The ICD Engine 515 may also use received conversation information from consultation input process 540, and history of the patient from the read history process 550 as input to generate an ICD using machine learning. The ICD Engine 515 may process the received information along with one or more ICD dictionaries to generate and ICD estimate to create a diagnosis report for review by the user. The ICD Engine 515 may create a diagnosis report and note for review by the user. Although not shown, the ICD Engine 515 may use machine learning modules, such as deep learning or a classifier module provided by the AI engine 570, to generate an ICD for the patient. The generated ICD may be determined for a current consultation with the patient, with a higher weighting of recent information from the medical record.

The dictionaries 520 provide various dictionaries, libraries, and patterns to model information.

AI engine components 600 will be described in relation to FIG. 6. The AI engine components 600 shows component processes that may form part of an AI engine 610 that may be used as part of the medical record processing system. The AI engine 610 is divided into seven component processes that may be further subdivided.

A machine learning process 620 has sub processed of a deep learning process 625 and a predictive analytics process 630 that may be used for the machine learning process 620. The machine learning process 620 may be used by the AI engine 610 to determine classifications or process data in an electronic medical record. One example of the machine learning process 620 may be to determine an ICD based on information present in the electronic medical record.

A natural language processing process 635 may be used by the AI engine 610 to process human speech, such as information provided by a speech process 655. The natural language processing process 635 includes translation process 640, a classification and clustering process 645 and an information extraction process 650 to provide the natural language processing.

The speech process 655 may take recorded speech and convert the speech to text using a speech to text process 660 or convert text to an audible output using a text to speech process 665. Typically, the speech to text process 660 allows audio, such as a recording from a microphone, to be processed by the medical record processing system. An expert systems process 670 can provide relevant medical information for the AI engine 610.

The AI engine 610 also has a planning process 675 providing planning, scheduling and optimization. A robotics process 680 may be used by the AI engine 610 when interaction with physical objects is required. The AI engine 610 may also include a vision process 685 that has sub processes of an image recognition process 690 and a machine vision process 695. The vision process 685 may be applied to images received by the medical record processing system or images captured by the medical record processing system.

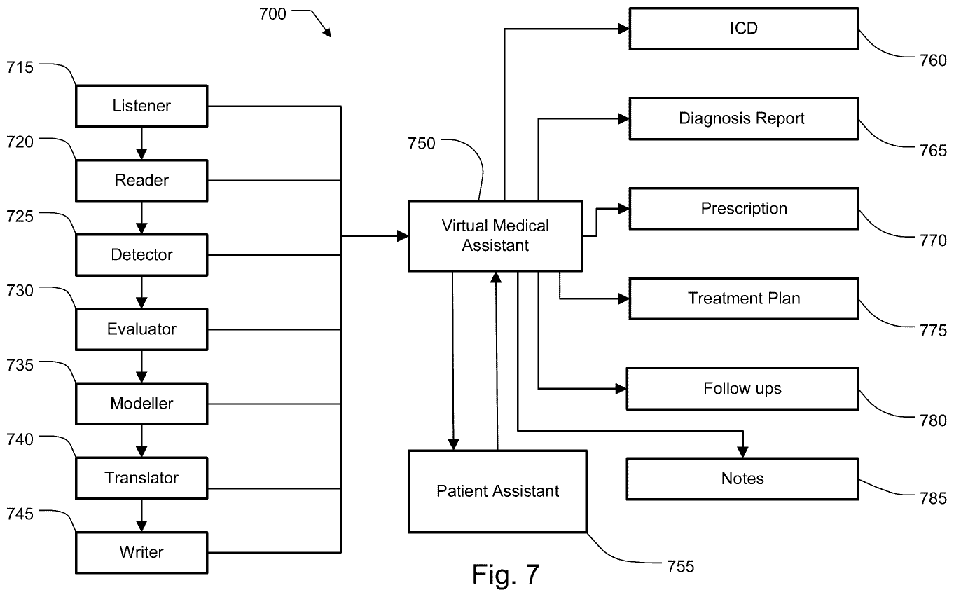

FIG. 7 shows medical record processing system communications 700. The medical record processing system communications 700 link modules and processes of the medical record processing system. Centrally located in the medical record processing system communications 700 is a virtual assistant 750 where data is collected from a number of data sources and used to generate a number of outputs. The medical record processing system communications 700 may be practiced on a computer such as the processing system 100 communicating over a network 202.

The medical record processing system communications 700 has a number of data collectors that can generate input to the virtual assistant 750. A listener 715 can process microphone inputs, when given permission by a user of the medical record processing system. A reader 720 processes text used in medical records and can pass the processed output to the virtual assistant 750. The reader 720 can process the historical information from a patient record so that the medical record processing system has access to historical information for the patient. The output of the reader 720, and other input modules, may be sent directly to the virtual assistant 750 or may be passed to other input modules for further processing before being sent to the virtual assistant 750.

The medical record processing system communications 700 has different types of input modules. One group of input modules is for further processing, where data from modules such as the listener 715 and the reader 720 may be further processed before being sent to the virtual assistant 750. The further processing modules include a detector 725, an evaluator 730, a modeller 735 and a translator 740. The detector 725 may process data to look for patterns and then extract the patterns for use by the virtual assistant 750. The evaluator 730 may compare readings in a patient file with expected values and report any discrepancies. The modeller 735 may look through the patient data and provide predictions into the future. In one example, the modeller 735 may look at blood test results and make a prediction of future blood test results. The translator 740 can process take input in one language and covert the input to another language. This allows the virtual assistant 750 to provide information to a user of the medical record processing system in their preferred language. A writer 745 may take data from other input modules and produce output that is suitable for presentation to a user, via the virtual assistant 750.

The virtual assistant 750 can produce a range of outputs related to the patient. In the medical record processing system communications 700 there are five outputs from the virtual assistant 750. Each of the outputs is determined by the virtual assistant 750 based on the input from the input modules and is specific to the patient. The virtual assistant 750 provides recommendations or suggestions to a user of the system, such as a clinician. The user may review the recommendations and accept, reject or modify the recommendations from the virtual assist ant 750. The virtual assistant 750 can output an ICD 760, a diagnosis report 765, a prescription 770, a treatment plan 775, a follow-up 780 or notes 785. The notes 785 may be clinician notes that the virtual assistant 750 generates based on data collected during a consultation, such a processing audio of a consultation between a patient and a clinician, collected using a microphone. The notes 785 may be presented to a user of the system for review before being accepted and added to the medical record of the patient. The follow-up 780 may include scheduling for follow up consultations or testing. The follow-up 780 may also include a scheduled process for the virtual assistant 750 to determine effectiveness of a treatment plan 775 compared to an estimated effectiveness. Not all of the recommendations are required for each visit for a patient. For example, a prescription 770 may not always be required and a treatment plan 775 or the follow-up 780 may not be required when treatment of the patient is simple.

Output from the virtual assistant 750 may send data to a patient assistant 755 and allow the patient assistant 755 to work with the patient. The patient assistant 755 may receive output from the virtual assistant 750, typically once the user of the medical record processing system has reviewed the output. Any output relevant to the patient may be sent to the patient assistant 755 to allow the patient assistant 755 to convey the information to the patient and provide follow-up information, reminders and monitoring. In one example, the prescription 770 is sent to the patient assistant 755 so that the patient can be reminded by the patient assistant 755 when the take their prescription medicine. Similarly, the treatment plan 775 may be sent to the patient assistant 755 so that any home treatment, such as exercises, can be communicated and monitored by the patient assistant 755. The patient assistant 755 may also provide reminders to the patient based on follow-up 780. The patient may access the patient assistant 755 via a web page on their computer or install an application on their smartphone to provide a user interface between the patient assistant 755 and the patient.

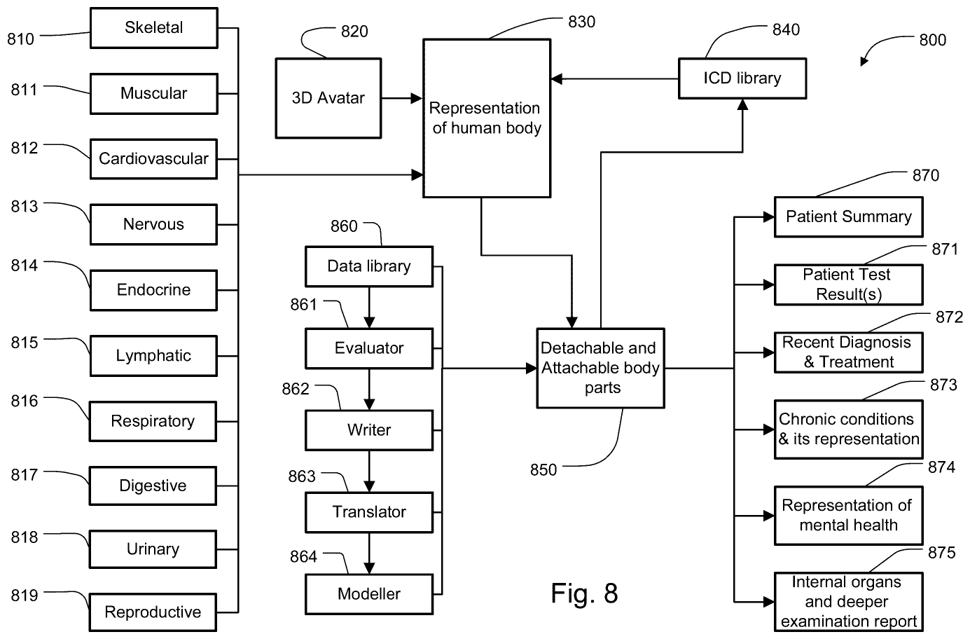

A 3D avatar process 800 will be described with reference to FIG. 8. The 3D avatar process 800 forms part of the medical record processing system and may be practiced on a computer such as the processing system 100 communicating over a network 202. Output from the 3D avatar process 800 is typically displayed to a user of the medical record processing system on a display to allow the user to have access to relevant medical information of the patient, organised using a 3D avatar.

The 3D avatar process 800 receives medical categories. In the example of the 3D avatar process 800 there are ten medical categories shown as skeletal 810, muscular 811, cardiovascular 812, nervous system 813, endocrine 814, lymphatic 815, respiratory 816, digestive 817, urinary 818 and reproductive 819. Patient data is assigned or categorised according to the medical categories using the AI engine by applying a classifier to the patient data. The medical categories are sent to a human body representation 830. The human body representation 830 also receives input from a 3D avatar process 820 and an ICD library 840. The information of the human body representation 830 is sent to a body parts process 850 where different information may be selected for display, as described above in relation to FIG. 3.

The 3D avatar 820 may operate in a similar way to the 3D avatar process 555 described above. The ICD library 840 provides relevant ICD (International Statistical Classification of Diseases and Related Health Problems) information that may be added to the human body representation 830.

The body parts process 850 also has as input a data library 860, evaluator 861, modeller 862, translator 863 and writer 864. The data library 860 may operate in a similar manner as the listener 715, reader 720, detector 725 described above, while the evaluator 861, modeller 862, translator 863 and the writer 864 operate similar to the evaluator 730, modeller 735, translator 740 and writer 745 described above. The input provides patient specific data that may be added to the data from the human body representation 830 in construction of the 3D avatar.

The body parts process 850 outputs a 3D representation of the patient that include the 3D avatar. The 3D representation may also include a patient summary 870, patient test results 871, recent diagnosis and treatment 872, a representation of chronic conditions 873, a representation of mental health 874 and internal organs 875. Some of the body parts process 850 outputs may be displayed to the user as part of the medical classifications associated with the 3D avatar, for example the internal organs 875, patient test results 871 and chronic conditions 873. Other parts of the output may be provided in addition to the 3D avatar, such as the patient summary 870 which may be displayed alongside the 3D avatar.

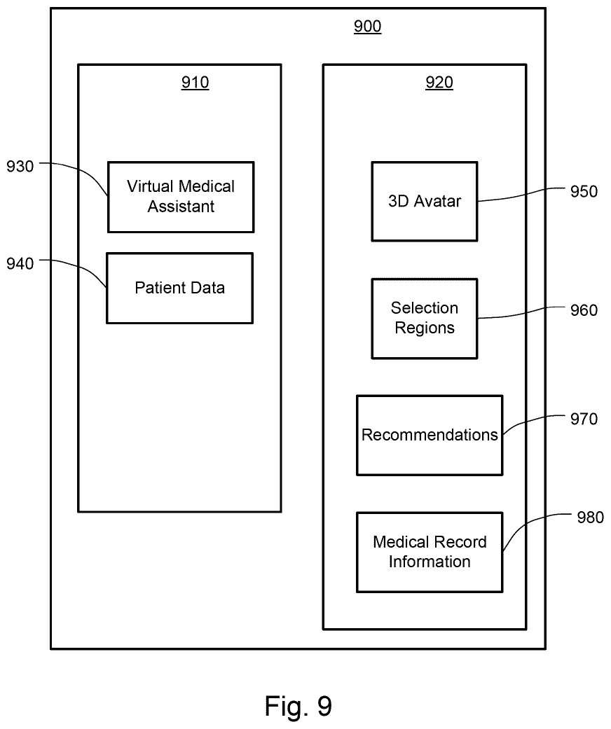

A medical record processing system 900 will now be described in relation to FIG. 9. The medical record processing system 900 may be practiced on a computer such as the processing system 100 communicating over a network 202. The medical record processing system 900 may have two parts, a processing system 910 and a display 920. The processing system 910 and the display 920 may be executed on a single computer or may be split over two or more computer or computing type devices. In one example, the processing system 910 may be executed on a remotely located server and the display 920 may be provided on a device such as a tablet, smart phone or smartwatch.

The processing system 910 includes a virtual medical assistant 930, that may operate as described above, as well as patient data 940. The virtual medical assistant 930 takes the patient data 940, processes the patient data 940 using modules such as an AI engine and sends the output to the display 920.

The display 920 will display a 3D avatar 950 that has selection regions 960. The selection regions 960 may be assigned to body parts of the 3D avatar 950, such as a leg, hand, arm or torso. The selection regions 960 may be assigned to biological system of the 3D avatar. Further medical record information 980, related to a medical category may be displayed when one of the selection regions 960 is selected by a user of the medical record processing system 900. The medical record processing system 900 allows the user to select one or more of the selection regions 960 using an input device such as a keyboard, mouse or touch screen. Each of the selection regions 960 is associated with, or corresponds to, one of the medical categories. Selecting one of the selection regions 960 will bring up and display medical record information 980 associated with the medical category corresponding to the selected region. By selecting one of the selection regions 960 a user of the medical record processing system 900 can browse the medical record of the patient using the 3D avatar 950 shown on the display 920. When a layered 3D avatar is displayed, such as the layered 3D avatar 370, selection subregions may be displayed.

The display 920 also includes recommendations 970 that are generated by the assistant process 930 and displayed to the user. The recommendations 970 may be as described above, in relation to FIG. 7, and include one or more of an ICD recommendation, a diagnosis report recommendation, a prescription recommendation, a treatment plan recommendation, follow ups recommendation and clinical notes recommendation. The recommendations 970 may be displayed in response to a user selecting a region or subregion, as a component of the display or as icons on the screen.

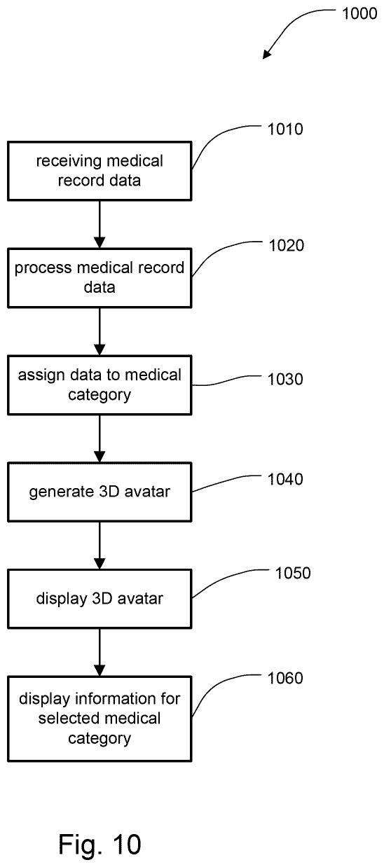

A medical record processing method, or process, 1000 will be described in relation to FIG. 10. The medical record processing method 1000 may be practiced on a computer such as the processing system 100 communicating over a network 202. The medical record processing method 1000 processes one or more medical records associated with the patient and presents information from the one or more medical record to a user of the system via a 3D avatar. The user is able to select regions of the 3D avatar to bring up more information relating to the patient. The regions of 3D avatar are associated with medical categories into which the information from the patient medical records is categorised.

The medical record processing method 1000 starts with a receiving process 1010 where data from one or more medical records for a patient are received from a source such as a database. Typically the medical records are in an electronic form. Alternatively, or in addition, the medical record data may be received from an audio input for a microphone recording a consultation between a clinician and a patient to receive data for a current medical problem. The medical data is then passed to a data processing process 1020 where information is extracted from the medical record data. The information from the medical record data is then assigned or categorised into a medical category at an assignment process 1030. The medical categories are described above. Typically, the assignment, or categorisation, of information from the medical record data of the patient, also referred to as patient data, is conducted by a machine learning module, such as a classifier, that processes the patient data of the medical record. The machine learning module is typically trained using medical record data where the information extracted from the medical record data been assigned to a medical category manually as training data.

An avatar generation process 1040 retrieves a 3D avatar from a database. The 3D avatar may be a generic representation of the patient that has selection regions associated with different portions of the 3D avatar. Each of the selection regions corresponds to at least one of the medical categories. Once the 3D avatar is displayed to a user, the user may select one of the regions to display more information from the medical record associated with the medical category. Subregions may also be displayed for a layered 3D avatar, such as the layered 3D avatar 370. At an avatar display process 1050 the 3D avatar from the avatar generation process 1040 is displayed to the user.

At a further information process 1060 the user may select one of the selection regions, or subregions, located on 3D avatar to select information from the medical record. Information assigned to the medical category corresponding to the region is displayed. In one example the user may select a region associated with a heart of the 3D avatar to bring up heart related medical data for the patient. Some parts of the medical record processing method 1000 may be repeated, such as the avatar generation process 1040 and the avatar display process 1050 to update the display of the 3D avatar. Diagnosis information may also form part of the avatar generation process 1040 and the avatar display process 1050 to display diagnosis information to the user.

### Variations

The medical records processing system described above may provide a display to user of the system on a computer, tablet, smart phone, smartwatch, wearable, clinician specific device, patient specific device or other display device. The user may control the medical record processing system using a wide range of input systems, such as a mouse, joystick, voice command, touch screen or gesture based control.

In one example, the layered 3D avatar may provide an accurate model of human anatomy, with all organs, etc. shown on various layers. The layers shown in FIG. 3B may be modified to show patient information with a first layer showing a patient summary as well as patient test results. A second layer may show recent diagnosis treatment. A third layer may show chronic conditions. A fourth layer may show a mental health and a fifth layer may show internal organs and deeper examination report. Alternatively, the layers may be shown as part of the 3D patient history 300 of FIG. 3A to provide a user of the medical record processing system with information on the patient.

While the described medical record processing system is shown as displaying medical record information to a user, the medical record processing system may also be used to capture data entry for the medical record. Once information related to a medical category or sub-category is displayed, the user may add more information to the category or subcategory. The additional information may be added by means such as text entry, attaching voice recording or images such as scan or photographic information. The added information is then recorded as part of the medical record of the patient.

The selectable regions may be positioned over a body part, biological system or some other part of the 3D avatar. In one example, all of the selectable regions are body parts or all of the regions are biological systems. In another example, the selectable regions are a combination of one or more body parts and one or more biological systems. In one example, the selectable regions are located over a body part or biological system for the corresponding medical category. Similarly, the medical categories may all be body parts or may all be biological systems. In another example, the medical categories may a combination of one or more body parts and one or more biological systems.

In one example of the layered 3D avatar 370 a heat map representation may be used to indicate an amount of medical information for a displayed item, such as a body part or biological system. For example, if the display item is knees and a patient has a lot of information related to their knees, then the knees on the bone layer 374 may be highlighted on the heat map. Examples of techniques to determine teg amount of information may be determined for the display item may include a number of entries, a number of tests, a number of consultations about the display item, a time duration over which there have been entries into the medical record for the display item, or a combination of two or more of the techniques. The clinician may navigate layers of the layered 3D avatar 370 while viewing the display item and may see detailed information for the patient. The heat may also be used on the non-layered 3D avatar, such as the 3D avatar 310.

### Advantages and Interpretations

The medical record processing system described provides a graphical interface for a user to browse through medical record information of a patient. The 3D avatar provided by the medical record processing system helps a user to visualize the data pictorially. By grouping the medical record information into medical categories the user of the system is able to locate relevant information related to the medical category quickly, without having to review all details of the medical record. The use of medical categories may help a clinician to reduce errors or missing formation when reviewing medical information for the patient by grouping relevant information together. The medical record processing system provides a patient centric model in the form of a 3D representation of the patient, such as the 3D patient history 300 or the layered 3D avatar 370, that may allow a clinician to understand a medical history of the patient. The medical record processing system may provide a better understanding of a patient, data visibility of each body part of the patient, indicators, heat map overlays which may assist the clinical to ensure wellbeing of the patient.

The medical record processing system may listen to a patient during a consultation and process the conversation between a clinician and a patient. The medical record processing system may create reports for the patient based on the conversation as well as information from the medical record of the patient. The medical record processing system may apply deep learning to the medical records of a patient and suggest treatment plans and steps for measuring treatment outcomes.

The medical record processing system described above provides a patient centric model to provide a user with information to improve understand a medical record of a patient. The use of AI modules, such as deep learning, allows the medical record processing system to provide recommendations to the user

The figures included herewith show aspects of non-limiting representative embodiments in accordance with the present disclosure, and particular structural elements shown in the figures may not be shown to scale or precisely to scale relative to each other. The depiction of a given element or consideration or use of a particular element number in a particular figure or a reference thereto in corresponding descriptive material can encompass the same, an equivalent, an analogous, categorically analogous, or similar element or element number identified in another figure or descriptive material associated therewith. The presence of “/” in a figure or text herein is understood to mean “and/or” unless otherwise indicated, i.e., “A/B”is understood to mean “A”or “B”or “A and B”.

Many modifications will be apparent to those skilled in the art without departing from the scope of the present invention.

The reference in this specification to any prior publication (or information derived from the prior publication), or to any matter which is known, is not, and should not be taken as an acknowledgment or admission or any form of suggestion that the prior publication (or information derived from the prior publication) or known matter forms part of the common general knowledge in the field of endeavour to which this specification relates.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

1. A system configured to display medical information about a patient, the system comprising:
a processor configured to process a medical record for the patient and assign information from the medical record to one of a plurality of medical categories; and a display for displaying a 3D representation of the patient, the 3D representation including a 3D avatar of the patient having corresponding regions for each of the plurality of medical categories,
wherein the processor receives an indication that a user has selected one of the regions and sends to the display information assigned to the corresponding medical category.

2. The system according to claim 1, wherein at least one of the medical categories is a biological system.

3. The system according to claim 1, wherein the medical categories are selected from the set consisting of: skeletal, muscular, cardiovascular, nervous system, endocrine, lymphatic, respiratory, digestive, urinary, mental health and reproductive.

4. The system according to claim 1, wherein the regions for the medical categories are representative regions of the 3D avatar.

5. The system according to claim 1, wherein the 3D representation is a layered architecture model of the 3D avatar.

6. (canceled)

7. The system according to claim 5, wherein the layered architecture model includes at least one layer selected from the set of: skin, muscular, organs and skeletal.

8. The system according to claim 5, wherein at least one of the regions is associated with at least one layer of the layered architecture model.

9. (canceled)

10. The system according to claim 7, wherein the at least one of the regions associated with the at least one layer is further divided in subregions and each of the subregions is associated with part of the at least one layer.

11. (canceled)

12. The system according to claim 10, wherein the at least one layer shows an icon to represent a diagnosis.

13. (canceled)

14. The system according to claim 1, wherein the information assigned to the corresponding medical category selected from the set of medical categories consisting of an ICD, a treatment plan and a treatment outcome.

15. (canceled)

16. (canceled)

17. A computer implemented method of displaying medical information, the method comprising:
receiving a medical record for a patient;
assigning information from the medical record to one of a plurality of medical categories;
displaying a 3D avatar for the patient, the 3D avatar including corresponding regions for each of the plurality of medical categories displayed on the 3D avatar; and
displaying the assigned information for one of the plurality of medical categories when a user selects the corresponding region.

18. The method according to claim 17, wherein each of the medical categories is a biological system.

19. The method according to claim 18, wherein the medical categories are selected from the set consisting of: skeletal, muscular, cardiovascular, nervous system, endocrine, lymphatic, respiratory, digestive, urinary, mental health and reproductive.

20. The method according to claim 19, wherein the regions for the medical categories are representative regions of the 3D avatar.

21. The method according to claim 17, wherein the 3D representation is a layered architecture model of the 3D avatar.

22. (canceled)

23. The method according to claim 21, wherein the layered architecture model includes at least one layer selected from the set of: skin, muscular, organs and skeletal.

24. The method according to claim 21, wherein at least one of the regions is associated with at least one layer of the layered architecture model.

25. (canceled)

26. The method according to claim 24, wherein the at least one of the regions associated with the at least one layer is further divided in subregions and each of the subregions is associated with part of the layer.

27. (canceled)

28. The method according to claim 23, wherein the at least one layer shows an icon to represent a diagnosis.

29. (canceled)

30. The method according to claim 17, to wherein the information assigned to the corresponding medical category is selected from the set consisting of an ICD, a treatment plan and a treatment outcome.

31. (canceled)

32. (canceled)
